(19) United States
(12) Patent Application Publication
(10) Pub. No.: US 2010/0094173 A1
(43) Pub. Date: Apr. 15, 2010

(54) PRESSURE CONTROL FOR CATHETER DRAIN TUBING

(76) Inventors: Marshall T. Denton, Salt Lake City, UT (US); Timothy R. Wolfe, Salt Lake City, UT (US); J. Michael Brown, Salt Lake City, UT (US); Perry W. Croll, Salt Lake City, UT (US); Mark A. Christensen, Salt Lake City, UT (US)

Correspondence Address: TRASKBRITT, P.C., P.O. BOX 2550, SALT LAKE CITY, UT, 84110, US

(21) Appl. No.: 12/450,123

(22) PCT Filed: Mar. 14, 2008
(86) PCT No.: PCT/US2008/003444

§ 371 Date: Sep. 11, 2009

Related U.S. Application Data
(60) Provisional application No. 60/918,386, filed on Mar. 16, 2007.

(51) Int. Cl. A61M 25/14; A61B 5/20

(52) U.S. Cl. 600/584; 604/323

(57) ABSTRACT

Described is, e.g., an apparatus (100), and associated methods of manufacture and use, for draining a fluid, discharged from a urinary catheter (104) installed in a subject's bladder, into a drain container (138) while avoiding undesired pressure states in such bladder. A drain conduit (102) connected to the catheter has a sufficiently small size as to maintain plug-flow between opposite ends of the drain conduit. The discharge end of the drain conduit is connected to a siphon-break apparatus (106) configured to cause a siphon-break disposed at an elevation (132) in approximate agreement with the subject's bladder. The potential energy from fluid downstream of the siphon-break location is decoupled from fluid in the drain conduit.

### PRIORITY CLAIM

This application claims the benefit under 35 U.S.C. §119(e) of the filing date of U.S. Provisional Patent Application Ser. No. 60/918,386, filed Mar. 16, 2007, for “PRESSURE CONTROL FOR CATHETER DRAIN TUBING”, the contents of the entirety of which are incorporated by this reference.

### TECHNICAL FIELD

The invention relates generally to medical devices such as low-pressure plumbing apparatus, and particularly to fluid-transporting structures arranged to transport fluid from a subject's bladder to a drain container.

### BACKGROUND

Various plumbing arrangements have been used for draining the discharge received from a catheter installed in a subject (e.g., a medical or veterinary patient). Two undesired drain pressure states are commonly found in certain conventional embodiments of known urinary catheter drain conduits: either excessive suction or excessive back-pressure. Such pressure states may be exerted on the bladder with potentially detrimental effect.

Excessive suction can be caused by a siphon effect imparted by a continuous column of urine extending below the level of the bladder in a discharge conduit. Such column is particularly likely in drain conduits having a sufficiently small bore. A sufficiently small bore promotes what may be characterized as plug-flow of discharge fluid in the conduit. A sufficiently small bore effectively resists breakdown of a meniscus disposed at a leading edge of a fluid filling the bore. In such a sufficiently small bore, surface tension of the fluid is sufficient to maintain the meniscus intact, and in contact with the circumference of the bore, despite gravitational forces at work on the fluid. A plumbing arrangement effective to determine a maximum bore size to ensure plug-flow includes an arrangement in which the meniscus traverses a vertically disposed radius of curvature in the conduit along a drain path, e.g., during a change from horizontal to vertical fluid flow. Excessive suction can cause pressure ulcers in the bladder by suctioning the bladder's mucosal wall up against the catheter's drain entrance port.

One source of excessive back-pressure includes a vertical section disposed in a catheter's discharge tubing that is not routed along a straight path from the catheter to the fluid collection container. Back-pressure may be caused during the first filling of a section of drain conduit that is routed to increase elevation of fluid therein with respect to the bladder. A pressure-inducing vertical section may also be defined by a leg of a U-shaped bend in a conduit having a sufficiently large bore. A sufficiently large bore permits conversion of plug-flow to particle-flow by permitting breakdown of the meniscus at the leading edge of a fluid filling the bore, e.g., as the meniscus encounters a rapid decrease in slope of the conduit along the drain path. Particle flow, or alternatively, detached stream flow, may even result during horizontal flow of fluid in a bore of sufficiently large diameter. In any event, particle-flow fluid often will coalesce at bottoms of U-shaped sections of drain tubing, change to plug-flow, and cause a back-pressure as the reformed plug of fluid is elevated during its escape from the U-shape.

Pressure exerted from the urine drain assembly is reflected in the pressure measured in the bladder. Excessive back-pressure can cause bladder distension and attendant patient discomfort. Bladder pressure may sometimes be measured and monitored as an indicator of abdominal pressure. Back-pressure from the drain assembly undesirably leads to inaccuracy in abdominal pressure indication, frustrating the ability to monitor an important medical parameter. Furthermore, back-pressure in the urine drain tubing assembly has an effect on bladder discharge, and may undesirably reduce measurement accuracy of a patient's urine output over time. Urine retained in the bladder for an extended period of time provides an undesirable nidus for infection.

Static pressure in an open drain conduit can be calculated, as is known, using potential energy of a fluid column. It is believed not uncommon for a patient having an installed commercially available urinary catheter to experience sustained intervals of back-pressure in excess of eight inches (20 cm) of water, or about 15-20 mm Hg, and sometimes more.

U.S. Pat. No. 2,602,448, issued Jul. 8, 1952, to McKenna, the contents of which are incorporated herein by this reference, discloses a plumbing arrangement including an automatic valve effective to cause periodic infusion and evacuation of an intubated patient's bladder. An elevated fluid source is adjusted to supply a slow fluid flow through the automatic valve toward the patient's bladder. Fluid flows into the patient's bladder, and simultaneously accumulates in the valve, until the bladder is full, at which time the valve's siphon element is charged by a cascade of accumulated fluid. The valve then fully evacuates the bladder using siphonic action, and leaves a water trap to resist air flow into the patient through the catheter.

U.S. Pat. No. 2,860,636, issued Nov. 18, 1956 to Seitchik et al., the contents of which are incorporated herein by this reference, discloses an automatic siphonic valve arrangement similar to that disclosed in the aforementioned >448 patent. Seitchik et al. disclose that common practice includes disposing the intake end of the siphon element at an elevation that is at least six inches (15 cm) below the bladder.

U.S. Pat. No. 3,598,124, issued Aug. 10, 1971, to Andersen et al., the contents of which are incorporated herein by this reference, discloses a plumbing arrangement for a urinary catheter drain conduit that is adapted to alleviate both excessive suction and excessive back-pressure. Andersen et al.'s plumbing arrangement is also adapted to cause cyclical filling of the bladder to a threshold pressure, followed by substantially complete bladder emptying, representative of natural operation of the urinary system. The threshold pressure can be adjusted by defining a desired difference in elevation between the highest point in their drain conduit and the bladder elevation. Their disclosed plumbing arrangement includes a pressure relief valve, disposed near the catheter and upstream of the highest point in the drain conduit. The pressure relief valve is adapted to admit air into the drain conduit subsequent to an emptying cycle of the bladder, and is actuated by siphon-induced suction. The admitted air acts as a hydraulic cut-off switch to allow the column of fluid downstream from the pressure relief valve to fully drain into a collection container, thereby removing both the siphon-induced suction and threshold pressure.

U.S. Pat. No. 6,736,803, issued May 18, 2004, to Cawood, the contents of which are incorporated herein by this reference, discloses a urine collection bag for use with a catheter and including a self-retracting drain tube adapted to empty the collection bag. The collection bag is adapted to be worn by a catheterized ambulatory patient, typically over the abdomen region. The drain tube includes a valve at its discharge end, which may be closed to resist spilling urine as the tube retracts subsequent to its use when emptying the collection bag.

Other relevant United States patents, which disclose structures adapted to facilitate fluid control during use of a urinary catheter include: U.S. Pat. Nos. 3,298,370; 3,800,795; 3,901,235; 4,343,316; 4,349,029; 4,981,474; 5,019,059; 5,429,624; 5,451,218; 5,505,717; 5,616,138; 6,311,339; 6,482,190; 6,500,158; 6,673,051; 6,740,066; 7,008,407; and 7,150,740 (the contents of each of which are incorporated herein by this reference).

Urine production from a patient is an indicator having medical importance and that is commonly recorded during routine care of a urine-catheterized patient. Current conventional practice includes periodically ensuring the drain conduit spanning between the catheter and drain container is fully emptied into the drain container. Mid-span portions of such drain conduit are typically elevated by a health care provider to promote complete drainage of any fluid entrapped in gooseneck portions of the drain path prior to recording the level of fluid held in the drain container. Such recordings are typically made at regular intervals, such as hourly. However, the requirement to manually evacuate standing portions of discharged fluid inevitably results in variably complete evacuation, and leads to uncertainty in the patient's actual urine output. Furthermore, elevating a mid-span portion of drain tubing may undesirably cause retrograde urine flow, which could contaminate the bladder.

Despite the numerous advancements made to date (e.g., as disclosed in the aforementioned patents), and the significant period of time during which various plumbing arrangements have been used for draining the discharge received from a urinary catheter installed in a medical patient, a need still exists for a robust drain arrangement that resists undesired positive and/or negative drain pressure, and/or severe fluctuations in such drain pressure.

### DISCLOSURE OF THE INVENTION

Embodiments structured according to certain aspects of the instant invention provide an apparatus, and method of use of that apparatus, for draining a fluid discharged from a medical patient's bladder into a drain container while avoiding undesired pressure states in that patient's bladder.

One operable assembly includes a siphon conduit disposed between a urinary catheter and a siphon-break device. The siphon conduit typically defines a fluid communicating lumen having a substantially constant cross-section disposed between its first end and second end. Desirably, the cross-section of the siphon conduit is configured and arranged to maintain plug-flow of a fluid flowing therethrough. In such case, and subsequent to a first filling with fluid, the siphon conduit is structured to maintain a substantially continuous column of fluid from its first end substantially to its second end. That is, any bubbles introduced at an entrance end of the siphon conduit, or off-gassed from the fluid flowing therethrough, will be transported to the discharge end without undergoing a meniscus breakdown that permits particle flow inside the siphon conduit.

Sometimes, a siphon conduit may include an extension structure to assist in tidy management of the conduit's path. One such extension structure includes a stretch of lumen coiled about an axis and adapted for extension along that axis effective to lengthen an effective drain path between the siphon conduit's first end and second end. Another operable extension structure includes a serpentine labyrinth of substantially in-plane curved flow sections adapted for extension by changing a length of lumen stretched between first and second curved flow sections. Similarly, a length adjustment may be effected in a serpentine labyrinth of such in-plane curved flow sections by changing a radius of curvature of at least one curved flow section. In certain cases, siphon conduit may be routed in combination with a hose management tool adapted to confine a serpentine labyrinth to a desired location in space. Desirably, such tool is configured for attachment to structure associated with a bed, to assist in disposing a siphon-break at a desired elevation. One operable such tool is adapted to permit transverse installation of a portion of the siphon conduit between gripping structure of the tool.

The siphon-break device is typically disposed at an elevation to cause a siphon-break to occur at a desired elevation with respect to a nominal location of the patient's bladder. The elevation of such siphon-break location may be selected irrespective of the elevation of fluid collected in a lower, or downstream, urine drain container. The siphon-break device, and any associated conduit disposed downstream of the siphon-break location, may be characterized as being effective to decouple the potential energy of fluid flowing downstream of the siphon-break (and along a path extending toward the drain container) from having an effect on fluid contained upstream of the siphon-break and in the siphon conduit. An operable siphon-break device includes a lumen having a cross-section that is larger than the cross-section inside of the siphon conduit and effective to promote particle-flow of fluid downstream from the siphon-break. In any case, a siphon-break device is desirably configured and arranged to cause particle-flow (or detached flow) commencing at a siphon-break location for a fluid flowing through the siphon-break device. The fluid flow path disposed downstream of the siphon-break location is typically arranged to define a path adapted to maintain particle-flow, or detached stream flow, of fluid between the siphon-break location and a container in which to receive drained fluid. Desirably, the drain path between the siphon-break location and container has a continuous downward slope.

In certain cases, a length of the siphon-break device (or alternatively, a drain conduit communicating from the siphon-break device) may be adjusted to accommodate a plurality of distances between the siphon-break location and drain container to permit disposing the siphon-break location at approximately the same elevation as a patient's bladder. Subsequent to completely charging the siphon conduit with fluid, the location of the siphon-break desirably controls the pressure at the entrance of a urinary catheter installed in the patient's bladder. Typically, the siphon-break is disposed within plus or minus 6 inches (15 cm) of a nominal bladder elevation of the patient. Preferably, the siphon-break elevation is disposed within positive 1 inch (2.5 cm) to about negative 6 inches (15 cm) of the patient's bladder elevation. In a more preferred arrangement, the siphon-break is disposed at an elevation to cause a substantially neutral or slightly negative pressure at the catheter's entrance, such as positive 1 inch (2.5 cm) to about negative 4 inches (10 cm) of the patient's bladder elevation. In a most desirable configuration, the siphon-break location is disposed to maintain the patient's bladder in an at least substantially evacuated state.

Sometimes, a shape of the siphon-break apparatus may be adjusted to accommodate a plurality of distances between a desired siphon-break location and a drain container. One operable shape for a siphon-break apparatus includes a telescopic structure adapted for extension along an axis effective to lengthen, or shorten, a drain path between the siphon conduit and drain container. Another operable shape includes a stretch of conduit coiled about an axis and adapted for extension, or retraction, along that axis. A further operable shape includes a serpentine labyrinth of substantially in-plane curved flow sections adapted for extension, or retraction, along an axis by changing a radius of curvature of at least one curved flow section.

In certain alternative embodiments, anchoring structure associated with the container is configured to permit making a vertical adjustment of the container with respect to suspension structure associated with the bed effective to facilitate disposing the siphon-break device at a desired elevation with respect to the bladder. Such embodiments avoid causing clinicians to perform a field operation including cutting a conduit to fit the spacing between a desired siphon-break location and an inlet to the drain container.

In some cases, a siphon-break device may be configured and arranged in harmony with the siphon conduit to visually indicate a corner approximately corresponding to the location of the siphon-break. It has been determined that the surface of the bed is a robust and reliable indicator of a desired location for a siphon-break to occur. Desirably, an anchor is also included, which is effective to maintain that corner in approximate registration with a top surface edge of a bed on which the drain apparatus may be installed.

A pressure indicator may sometimes be disposed in-circuit, desirably in relatively close proximity to the siphon-break device and in fluid communication with the siphon conduit. Such an indicator permits a clinician to visually verify a desired pressure state in drained fluid and near the siphon-break location. Feedback from such pressure indicating device may indicate a need to make an adjustment to the plumbing apparatus, including correction of the elevation of the siphon-break location with respect to the nominal location of the patient's bladder.

A method for placing a urinary catheter in fluid communication with a drain container using an embodiment structured according to certain principles of the instant invention includes: first, installing a urinary catheter in position to drain a patient's bladder. Second, a first end of a siphon conduit is placed into fluid communication with a discharge end of the catheter, with the siphon conduit being constructed to maintain plug-flow of a fluid flowing therethrough. Third, the siphon conduit is routed along a path disposed at approximately the same elevation to dispose the siphon conduit discharge end elevated above a drain container and to cause a siphon-break at an elevation in general agreement with the bladder. The siphon conduit may be routed over a leg, but generally follows the top surface of the bed in which the patient is disposed. In most cases, such siphon-break elevation desirably is selected to cause a small (e.g., 1 to 3 inches (2.5 to 8 cm) of water column) negative pressure in the bladder to promote complete evacuation. Most preferably, a substantially neutral, but slightly negative, pressure is induced (e.g., 1 to 2 cm water column). However, a small or neutral positive pressure may also be induced in certain alternative cases. Fourth, a first end of a siphon-break device is placed into fluid communication with the discharge end of the siphon conduit, where the siphon-break structure is constructed to resist plug-flow of a fluid flowing therethrough. And fifth, a second end of the siphon-break device is placed in fluid communication with the drain container.

In a method of using a drain apparatus structured according to certain principles of the instant invention, a urinary catheter is installed in a patient. A first end of a siphon drain conduit is placed in fluid communication with a discharge end of the catheter. A siphon-break apparatus, connected to the other end of the siphon conduit, is anchored at an elevation with respect to the bladder effective to induce a pressure between about positive 1 inch (2.5 cm) water column and about negative 6 inches (15 cm) water column at a drain entrance of the catheter. The discharge from the siphon-break apparatus is then placed in fluid communication with apparatus adapted to measure fluid flow rate.

In certain cases, the apparatus adapted to measure fluid flow rate may include an automated fluid flow rate determining device, such as a droplet counter. An alternative automated fluid flow rate determining device may include a data acquisition system adapted to estimate flow rate by measuring a difference in discharged fluid weight in one or more time periods, and performing appropriate conversion calculations.

In an alternative case, an embodiment of a drain apparatus adapted to manually measure discharged fluid flow rate may include a first container adapted to indicate a volume of fluid held therein. In the later case, the method may further include waiting for a first period of time during which a charge-volume at least fills drain structure disposed between the bladder and a siphon-break, and determining a first volume contained in the first container at the conclusion of the first period of time. After waiting for a second period of time, a second volume contained in the container at the conclusion of the second period of time is determined. Then, a flow rate can be calculated using the difference between first and second volumes divided by the second period of time. Of course, the last two steps can be repeated, with the flow rate being determined by dividing the difference in discharged volume by the corresponding time increment in which such volume was discharged. The measured volume at the end of each time period may sometimes be transferred to a bulk container. In such case, the fluid output can be calculated by dividing each measured volume by the period of time in which it was collected.

### BRIEF DESCRIPTION OF DRAWINGS

FIG. 1 is a schematic view in elevation of a drain assembly structured according to certain principles disclosed here in.

FIG. 2 is a plan view in elevation of an alternative configuration for a siphon-break apparatus.

FIG. 3 is a plan view in elevation of another alternative configuration for a siphon-break apparatus.

FIG. 4 is a close-up view, in section, of a portion of siphon-break apparatus.

FIG. 5 is a view in elevation of a drain assembly structured according to certain principles of the instant invention associated with a bedridden patient.

FIG. 6 illustrates an arrangement adapted to manage an effective length for a stretch of drain conduit used in certain embodiments structured according to certain principles described herein.

FIG. 7 is a cross-section view, taken through section 7-7 in FIG. 6, and looking in the direction of the arrows.

FIG. 8 is a schematic view in perspective of a portion of a drain assembly installed on a patient's bed.

FIG. 9 is a cross-section view of a workable siphon conduit.

FIG. 10 is a cross-section view in elevation of a currently preferred siphon-break and an associated pressure gage.

### BEST MODE(S) FOR CARRYING OUT THE INVENTION

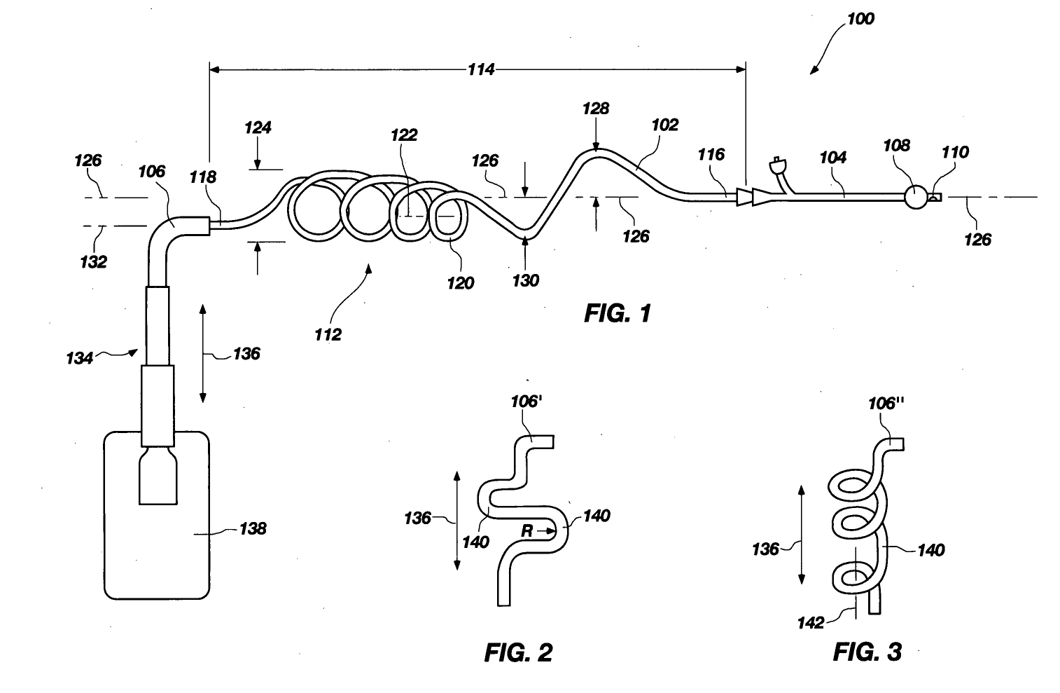

A first embodiment of a catheter drain assembly structured according to certain principles of the instant invention is illustrated in FIG. 1, and is indicated generally at 100. Catheter drain assembly 100 includes a draining siphon conduit 102 disposed between a urinary catheter 104 and a siphon-break apparatus 106. Illustrated catheter 104 is a conventional dual-lumen catheter including a retention balloon 108 carried at its distal end in proximity to the drain aperture 110, or entrance. Other urinary catheters, including single-lumen catheters and catheters having a further plurality of lumens, may be used in alternative workable arrangements structured according to certain principles of the instant invention.

Conduit 102 desirably includes extension structure, generally indicated at 112, operably arrangeable to change an effective length 114 between its first end 116 and second end 118. Extension structure of some sort is desirable to assist in tidy management of the drain path provided by conduit 102, and to resist routing of such path in a configuration that inherently produces an undesired pressure state in the patient's bladder. While other shapes and sorts of conduit structures are workable, conduit 102 is typically formed from a stretch of smooth-bored medical grade tubing providing an internal lumen with an approximately round cross-section bore.

The extension structure 112 illustrated in FIG. 1 includes a plurality of coils 120 that may be characterized as being coiled about an axis 122, and adapted for extension, or retraction, along axis 122 to change effective length 114. In practice, because the conduit 102 desirably is compliant to promote patient comfort, such coils 120 having a sufficiently large diameter 124 typically may “tip over” from their illustrated approximately vertical position, to a substantially horizontal spiraling position in general agreement with the top surface of the bed.

An extension structure 112 may be arranged to form a portion of the conduit 102, or may essentially encompass the entire stretch of such conduit. In one configuration within contemplation, certain coils 120 may be self-biased toward a shorter length, and “stretched-out” to fit, as desired to accommodate a particular patient and bed arrangement. In another configuration, adjacent coils may be adhered to each other to form a compact group, and a desired stretch 114 may be accommodated by separating a sufficient length from the adhered group of coils 120. Such separated length may be stretched and substantially straightened, or may remain biased toward a coiled shape. Certain embodiments of workable extension structure may be, or include portions that are, plastically deformable, to assist in maintaining a certain shape and total length that may be imposed by a health practitioner. In such latter case, a siphon conduit 102 may advantageously be arranged so as to axially follow the course of the patient's leg, then be bent to transversely progress toward the edge of the bed. Sometimes, a malleable length of wire may be included in a conduit to help the conduit hold a desired deformed shape.

With reference still to FIG. 1, the nominal elevation of the patient's bladder is indicated by interrupted centerline 126. In practice, conduit 102 may be routed around and over portions of a patient, creating stretches that are elevated 128, and stretches that are lowered 130, compared to the bladder elevation 126. Typically, the difference in elevation of any stretch between first end 116 and second end 118 is desirably held to between about plus 6 inches (15 cm) to about minus 6 inches (15 cm).

Fluid moves sufficiently slowly in conduit 102 during normal use that, as a practical matter, friction between the tubing and fluid may be disregarded when calculating pressures. However, as the conduit is first charged, stretches 128, 130 may, temporarily, have an effect on pressure exerted in the patient's bladder. As the conduit 102 is being initially charged, the corresponding pressure exerted in the patient's bladder will fluctuate as the leading edge of plug-flow fluid changes elevation relative to the bladder. Siphonic action as the leading edge of the column of fluid moves lower than the bladder causes a negative pressure, or suction, at the bladder. A positive pressure, or back-pressure effective to resist fluid flow from the bladder, is generated as the leading edge of the column of fluid in conduit 102 moves above the bladder's elevation 126.

The siphon conduit 102 may be manually charged to avoid, or minimize, holding time during siphon-induced pressure fluctuation in the patient's bladder during initial charging of such conduit. Such manual charging may conveniently be done using one fluid-communicating lumen of a three (or more)-way urinary catheter.

Once the conduit 102 is fully charged with fluid, the pressure exerted in the patient's bladder due to fluid in conduit 102 is desirably controlled entirely by the difference between the bladder elevation 126 and approximately the elevation of second end 118. It is currently preferred to induce a small negative pressure at drain aperture 110 to promote complete emptying of the bladder, such as by maintaining second end 118 at an elevation to cause a siphon-break elevation 132 that is about 2 to 4 inches (5 to 10 cm) lower than bladder elevation 126.

With continued reference to FIG. 1, it is desirable for siphon-break apparatus 106 to include, or communicate with, drain extension structure, such as the telescopic portion indicated generally at 134. An operable extension structure permits making an adjustment in length, as indicated by two-headed arrow 136, between second end 118 and a drain container 138 in which to collect fluid discharged from the patient. Telescopic portion 134 can be formed from a plurality of conduit sections sized to slide with respect to each other, while maintaining a fluid resistant seal to contain fluids between second end 118 and the container 138.

Virtually any sort of drain container 138 may be used, so long as a suitable coupling with the discharge end of the siphon-break apparatus may be made. However, it is currently preferred that a drain container will include appropriate markings to facilitate monitoring the quantity of fluid discharged by the patient. Periodic monitoring of incremental such quantities permits calculation of a patient's fluid discharge rate. It is also preferred that the drain container 138, or some other structure included in the drain assembly, will provide a suitably protected vent, or other arrangement of structure, which is constructed to resist pressure build-up inside the container as fluid is discharged by the patient.

Alternative siphon-break structures 106′ and 106″ that include extension structure are illustrated in FIGS. 2 and 3, respectively. Each Of siphon-break structures 106, 106′, and 106″ may be characterized as generally defining a vertical (or at least continuously downward sloping) flow path between second end 118 and a fluid container, such as container 138. It is within contemplation for a drain conduit to be plastically deformable to permit making shape adjustments to the conduit effective to fit an effective length of drain conduit into the spacing between the siphon-break location and drain container. One operable plastically deformable drain conduit includes a length of malleable wire associated with that conduit.

Siphon-break structure 106′ includes a serpentine labyrinth of substantially in-plane curved flow sections 140 adapted for extension along an axis, as indicated by two-headed arrow 136, by changing a radius of curvature “R” of at least one of the curved flow sections to vertically lengthen or shorten a drain path between second end 118 and container 138. Siphon-break structure 106″ includes a stretch of conduit 140 coiled about an axis 142 and adapted for extension or retraction along axis 142, as indicated by two-headed arrow 136, effective to vertically lengthen or shorten a drain path between second end 118 and container 138. In any case, it is preferred that, downstream of the elevation 132 of the siphon-break itself, a continuous downward-sloping fluid drain path is provided, to resist pooling of fluid and consequent re-establishment of plug-flow, prior to emptying into container 138.

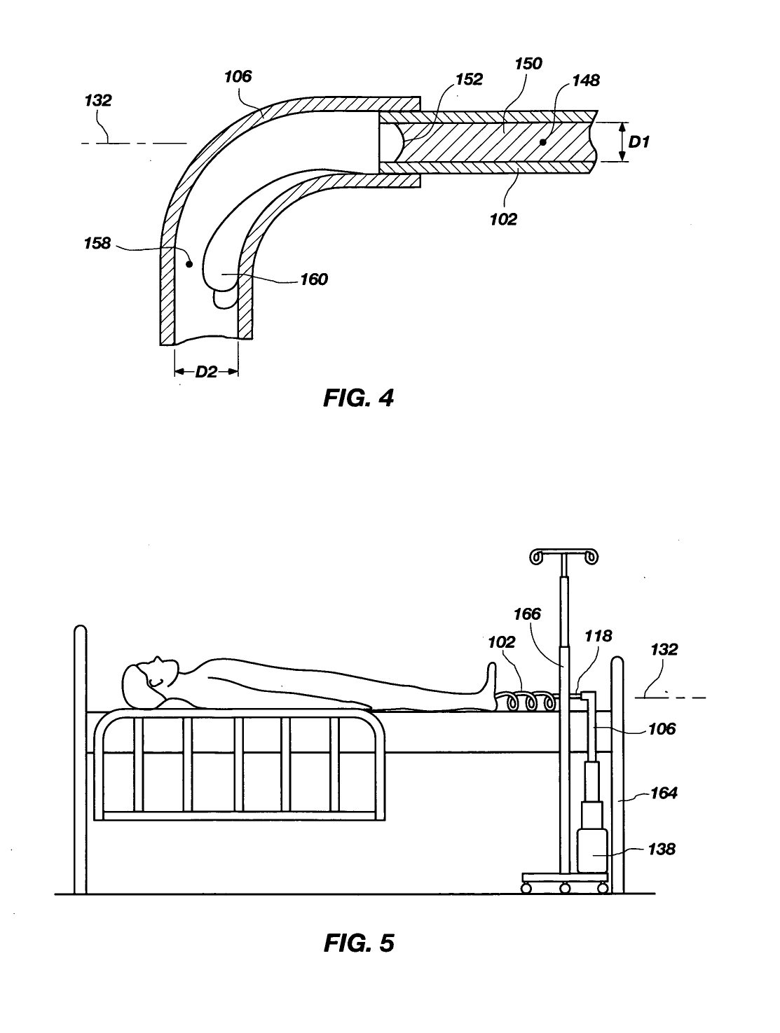

With reference now to FIG. 4, the size D1 of the bore 148 inside siphon conduit 102 desirably is selected to cause plug-flow of fluid 150 traveling along the lumen 148. For purpose of this disclosure, plug-flow means that the fluid fills the entire cross-section of the lumen, and in most cases, a meniscus 152 defining a front boundary wall of the “plug” is maintained intact by surface tension of the fluid. For example, in the case of a motionless plug of fluid contained inside an end section of tubing that is disposed in a vertical orientation, the meniscus 152 forming the leading edge of the plug (and spaced apart from the tubing's open end) will remain intact, and fluid will not drip from the tubing's open end. In practice, an operable upper size for a diameter effective to promote plug-flow of urine is less than about ¼ inches (6.3 mm), with an internal bore 148 size of less than about 3/16 inches (4.8 mm) being currently preferred to form a robust siphon conduit 102. In certain cases, lumen 148 having significantly smaller diameters are subject to occlusion from debris discharged from certain patients.

Still with reference to FIG. 4, siphon-break apparatus 106 includes a fluid-conducting bore 158 with a size D2 that is sufficiently large as to resist maintaining plug-flow of fluid flowing from conduit 102 as such fluid continues along the siphon-break structure 106 toward container 138. Instead, fluid 160 flowing through structure 106 may be characterized as comprising particle-flow, or perhaps stream flow. Such particle-flow, or stream flow, fails to fully occlude the bore 158, and therefore does not exert a pressure influence on the fluid 150 inside siphon conduit 102. Put another way, the potential energy of fluid 160 inside siphon-break structure 106 is desirably decoupled from fluid 150 inside siphon conduit 102 subsequent to the siphon-break event. Such decoupling results from conversion of plug-flow to particle-flow by the siphon-break structure 106. The siphon-break elevation may be selected irrespective of the elevation of fluid collected in a urine drain container 138. Of course, it must be appreciated that fluid downstream from the siphon-break is generally not permitted to pool and occlude the drain path between the siphon-break elevation and the drain container, or such pooled fluid could undesirably impact on the pressure in the patient's bladder.

As illustrated in FIG. 4, an operable siphon-break apparatus 106 may be formed from a medical grade plastic, or plastic-like, conduit having an inside diameter sized to couple with the outside diameter of siphon conduit 102 to form a union between such drain structures. It should be recognized that various known coupling devices may alternatively be interposed at such union to couple a larger sized siphon-break apparatus to the siphon conduit. Furthermore, an operable siphon-break apparatus 106 may be formed as an assembly of alternative constituent components that are effective to cause a siphon-break, and desirably, to resist plug-flow between such break and a drain container.

With reference now to FIG. 5, it is preferred to maintain an elevation of the siphon-break 132 by anchoring second end 118, or structure associated with end 118, to anchor structure that is not likely to change in elevation, such as foot rail 164 of the bed, or the vertical extension pole of stand 166. Such anchoring may be accomplished using various anchoring devices known in the art, including adhesive tape, length of twine, rope, or cord, twist-tie devices, mechanical clamp mechanisms, and the like.

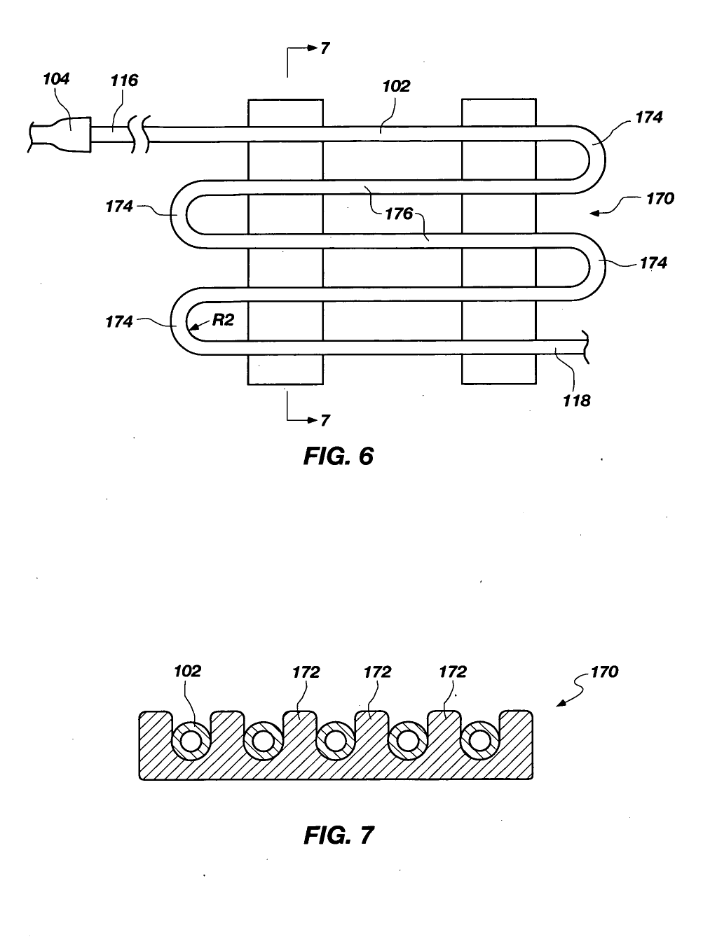

FIGS. 6 and 7 illustrate details of an alternative arrangement, generally indicated at 170, for maintaining tidy order in the routing of conduit, such as siphon conduit 102. Conduit management system 170 includes a plurality of fingers 172 between which conduit 102 may be held. Stretches of conduit 102 may be installed in one or more tool 170 by applying a transverse displacement to nestle the conduit 102 between adjacent fingers 172. As illustrated, the conduit 102 may be routed between cooperating groups of fingers 172 to form a serpentine labyrinth of substantially in-plane, curved flow sections 174 spaced apart by variable stretches of lumen 176 spanning between adjacent curved flow sections. The size of one or more stretch 176 can be adjusted to lengthen, or shorten, an effective drain path size 114 between first end 116 and second end 118. A similar length adjustment to drain path size 114 may also be made by changing a radius of curvature R2 of one or more curved flow sections 174.

Organizing tool 170 may also serve as an anchor from which to locate the position for siphon-break elevation 132. Anchor structure (not illustrated) may be included in an association with certain tools 170 to assist in attaching a tool 170 to support structure, such as a bed rail 164, or stand 166. Such anchor structure may be adapted to orient the one or more anchored tool 170 in space effective to maintain the portion of conduit 102 associated with such tool(s) within a desired range of elevation.

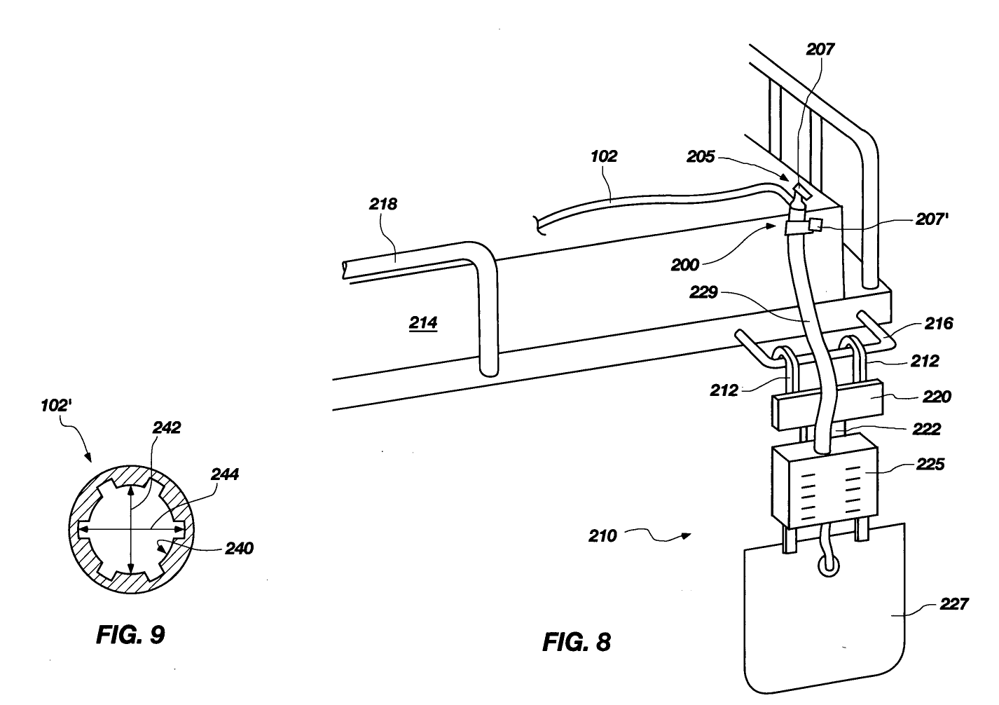

In certain embodiments, the siphon-break device is configured and arranged to visually indicate an approximate location (and therefore elevation 132, FIG. 1) of the siphon-break. As illustrated in FIG. 8, the siphon-break device, generally 200, forms a corner at its connection to the siphon conduit 102. Such a corner at least approximately identifies the siphon-break location, and may therefore assist a health practitioner to dispose the siphon-break location at a desired elevation with respect to the bladder of a patient. Desirably, a local anchor, generally 205, is included to maintain the siphon-break location in approximate registration with a top surface edge of a bed on which the bladder-draining apparatus is installed. It is believed that the corner formed in the device 200 provides a visual clue that naturally urges a health practitioner to place the siphon-break location in agreement with the edge of a patient's bed.

With continued reference to FIG. 8, an operable local anchor 205 includes one or more biased clip 207 that may be affixed at one end to the siphon-break device, and at an opposite end to a sheet or other part of the bedding. Illustrated clip 207 is somewhat like a clothespin, and can clamp onto a folded portion of bedclothes, such as a sheet or blanket. Alternative structure effective to form a local anchor for a siphon-break device may attach at an opposite end to the bed, bed-rail or other bed structure, or even the floor. As illustrated in FIG. 8, at least a portion of the local anchor 205 can be affixed at one end directly to the siphon-break device, or to an adjacent section of conduit.

Still with reference to FIG. 8, it is often desirable to couple a urine discharge meter, generally indicated at 210, to the drain conduit downstream of the siphon-break location. Illustrated urine discharge meter 210 is typically suspended from a patient's bed. The illustrated suspension structure includes a pair of hooks 212 configured for attachment to suspension structure associated with a bed 214, such as adjustment bar 216, or fence 218.

Hooks 212 suspend a base 220 and rail 222 at an elevation relative to the bed. A urine meter 225 is carried on the rail 222, and a bulk collection bag 227 is carried by the meter 225. After recording the volume collected in the meter during a first period of time, a valve may be manually opened to drain the measured fluid into the bag 227. Then, the valve is closed to permit collection and measurement of fluid in meter 225 for a subsequent period of time. In such case, the fluid output can be calculated by dividing each measured volume by the period of time in which it was collected. A similar and operable urine meter is available from Welford Medical, Inc., and includes a urine meter with a 500 mL measuring chamber and a 2000 mL downstream bulk collection bag.

In some cases, the hooks 212 may be vertically adjusted, relative to the base, to variably space the base 220 apart from the suspension structure. In some cases, the meter 225 is vertically adjustable along the rail to variably space the meter apart from the suspension structure. In any case, it is desirable to provide some adjustment in mounting the drain container to permit locating the siphon-break at a desired elevation with respect to the patient's bladder, and to maintain the drain conduit portion 229 in a configuration to resist formation of plug flow in fluid downstream of the siphon-break. Of course, the length of conduit 229 may simply be cut-to-fit in the field, to fit a particular installation. However, it is generally preferred to provide a urine drain assembly in pre-assembled kit-form, which requires minimal field assembly and is adjustable to accommodate anchoring a siphon-break location relative to different bed suspension structures.

A characteristic bore size of conduit 102 is generally determined by conflicting constraints. It is good for the characteristic size to be small as possible, to reduce the volume of urine contained between first and second ends of the conduit 102. A reasonable minimum characteristic cross-section size for a smooth-bore drain lumen might be about 0.040 inches (1 mm). In contrast, it is necessary for the characteristic size to be large enough to resist flow-blocking clot formation from debris build-up inside the drain lumen. An upper bound for a characteristic cross-section size is that size which is no longer capable of maintaining an intact meniscus at the fluid front as the fluid flows along a flow path that is curved from horizontal to vertical (or the size at which the lumen fails to maintain plug flow of fluid through the siphon conduit 102). It has been determined that a smooth-bore conduit sized about 0.160 inches (4.1 mm) is a compromise upper bound (including a margin of safety), beyond which particle flow may occur in a sterile saline reference fluid.

It is within contemplation to provide a siphon conduit 102 that has antimicrobial properties to resist build-up of biofilm and increased lubricity inside the drain lumen. An increase in lubricity corresponds to a decreased characteristic cross-section size capable of maintaining an intact meniscus. Therefore, an antimicrobial effect may serve to maintain a margin of safety against particle flow occurring in a siphon drain lumen. The antimicrobial property may be caused by application of a film agent to the lumen interior, or arise as a natural material property inherent in the conduit 102.

With reference now to FIGS. 1 and 9, a characteristic size of a lumen cross-section for siphon draining conduit 102 can be maximized to resist drain plugging, but still maintain a margin of safety against particle flow. One way to maximize a lumen size is by providing a lumen that has an alternative cross-section shape arranged to increase the surface area of the cross-section. The increased surface area helps to promote plug flow of fluid by resisting detachment of the meniscus from the lumen wall. One such alternative shape is illustrated by the conduit, generally indicated at 102′, in FIG. 9. The cross-section of conduit 102′ includes a plurality of inwardly projecting arms 240 that collectively define a minimum diameter 242. The space between arms 240 defines a maximum diameter 244. In one currently preferred conduit 102′, minimum diameter is about 0.120 inches (2.8 mm), and maximum diameter 244 is up to about 0.160 inches (4.1 mm), with a preferred maximum diameter size of about 0.150 inches (3.8 mm). One advantage provided by a cross-section shape such as suggested by FIG. 9 is an inherent increased resistance to kinking of the conduit 102′.

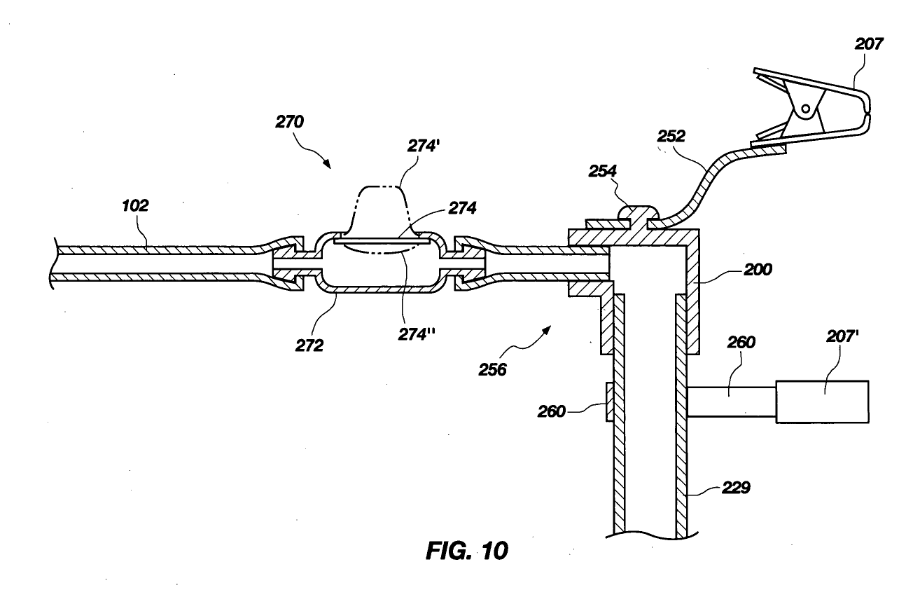

FIG. 10 illustrates an operable configuration to associate a local anchor with the a siphon-break device 200. A biased clip 207 is affixed directly to the housing portion of siphon-break device 200 by way of strap 252, which is secured by nub 254. Of course, there are many other ways in which to associate a local anchor with the siphon-break device that will be apparent to one of ordinary skill in the art. In use, strap 252 may be placed on the top of the mattress, and its associated clip 207 can be fastened to a sheet or blanket to dispose the corner, generally 256, substantially in transverse registration with the top edge of the mattress. One or more additional clips 207 may be included, and may be clipped to the sheet or blanket to resist longitudinal movement of the siphon-break device 200. As illustrated, an additional clip 207′ is carried on an end of a strap 260 that is wrapped around the drain conduit 229. Clip 207′ can conveniently be installed to grab a wrinkle formed in the sheet or blanket on the side-edge of the mattress to resist longitudinal movement of the siphon-break device 200 (e.g., see FIG. 8). Additional clips, or alternative anchoring devices, may be provided as required for a particular installation.

FIG. 10 also illustrates a pressure indicator, generally indicated at 270, that may be included in the drain assembly, desirably in proximity to the siphon-break device 200. Pressure indicator 270 may be used to verify lack of a downstream occlusion, properly venting drain container, and/or a desired negative pressure state. As illustrated, pressure indicator 270 presents certain desired operational characteristics, but is not rendered in absolute fidelity.

Because operating pressure of the fluid in the conduit 102 is very low during normal operation, the indicator 270 desirably provides a mechanical advantage by way of its larger working surface area, compared to the cross-section of drain conduit 102. It is within contemplation to couple small displacement of the large membrane area with a smaller cross-section to amplify travel of an indicator (not illustrated).

With continued reference to FIG. 10, transversely flexible membrane 274 is installed to provide a working surface and to resist undesired escape of fluid from the housing 272. If the fluid pressure inside indicator 270 is too high, the membrane 274 will displace to a position indicated by phantom line 274′. When the pressure is too high, a clinician should clear obstructions, check adequacy of container vent, or lower the elevation of the siphon-break. Conversely, if the pressure at indicator 270 is negative, the membrane 274 will displace to the position indicated by phantom line 274″. If the pressure is too low, the clinician should typically increase the elevation of the siphon break, to avoid causing a suction-induced pressure ulcer in the patient's bladder.

An embodiment structured according to certain principles of the instant invention may be used in a method for placing a urinary catheter in fluid communication with a drain container. One such method includes installing a urinary catheter 104 in position to drain a patient's bladder. A first end 116 of a siphon conduit 102 is then placed into fluid communication with a discharge end of the catheter 104. The siphon conduit 102 may be characterized as being constructed to maintain plug-flow of a fluid 150 flowing therethrough. The bulk of the length of siphon conduit 102 is typically routed along a path disposed at approximately the same elevation to dispose the siphon conduit discharge end 118 elevated above a drain container 138 and to permit a siphon-break to occur at an elevation 132 in general agreement with the bladder's elevation 126. In most cases, such siphon-break elevation 132 desirably is selected to cause a small (e.g., 1 to 3 inches (2.5 to 7.5 cm) of water column, or more preferably, 1 to 2 cm water column) negative pressure in the bladder to promote complete evacuation. A siphon-break apparatus 106 is placed into fluid communication with the discharge end 118 of the siphon conduit 102. A currently preferred and operable siphon-break structure 106 is constructed to cause the siphon-break to occur at a desired elevation 132 and to resist plug-flow of a fluid 160 flowing further therethrough. The drain end of the siphon-break apparatus 106 is placed in fluid communication with the drain container 138. The method may include anchoring structure associated with the siphon-break apparatus 106 effective to dispose a siphon-break at any desired elevation 132.

Subsequent to complete fluid-filling of that portion of the drain conduit assembly disposed between the patient and the siphon-break apparatus 106, the siphon-break elevation 132 determines the amount of either back-pressure, or suction, imposed on the bladder by the instant drain assembly. The pressure effect of the drain assembly may be regarded as being applied to the bladder at the drain orifice of an installed catheter 104. Currently, it is preferred for siphon-break elevation 132 to be maintained between about 1 inch (2.5 cm) above, to about 4 inches (10 cm) below, the elevation of the patient's bladder. In theory, it is most desirable to position the siphon-break at an elevation to maintain a neutral pressure, or a substantially neutral negative pressure (e.g., 1 to 2 cm water column), at the catheter's drain entrance in the bladder to promote complete evacuation of the bladder. In practice, the elevation of a patient's bladder may be difficult to determine, and the patient may change position and consequent bladder elevation. Therefore, it is presently preferred to dispose siphon-break elevation 132 effective to err on the side of inducing negative pressure, to promote complete evacuation of the bladder and to permit measurement of bladder pressure that is truly reflective of abdominal pressure.

Bladder pressure can be measured, even real time, while draining urine from a bladder, as disclosed in United States patent application by Sugrue et al., Ser. No. 11/219,316, titled “Apparatus for monitoring intra-abdominal pressure,” filed Sep. 1, 2005, the contents of which are incorporated herein by this reference. A urine drain assembly structured according to certain principles of the instant invention permits measuring bladder pressure to infer abdominal pressure while permitting uninterrupted flow of urine draining from the patient's bladder. In such case, “uninterrupted flow” means that the drain tubing, such as the siphon tube 102, is not occluded as a condition to permit making a pressure measurement. It is recognized that the flow of fluid in the conduit 102 may cease due to lack of fluid entering the Catheter's drain orifice when the bladder is substantially empty (and the catheter's drain entrance may even be occluded by bladder wall structure), but such is not regarded as “interrupting the flow” within the context of this method of using the instant drain apparatus. In contrast, “interrupting the flow” requires a fluid flow-resisting device, such as a hose clamp, or valve, typically operated by a health care worker, effective to stop fluid flow through the drain conduit 102.

Interestingly, a positive pressure, or back-pressure caused by the drain assembly, would be added to the patient's abdominal pressure (in a pressure measurement made in the bladder), because fluid in the bladder would communicate such pressure between the drain system and measurement system. However, a negative pressure, or suction, does not necessarily effect measured bladder pressure, because the drain orifice (e.g., 110, FIG. 1) can simply become plugged by structure of an empty bladder, such as a portion of the mucosal wall. Therefore, the pressure in the drain system can be separated, or decoupled, from communication with pressure in the measurement system. However, it is desirable to minimize the amount of applied negative pressure (e.g., to at least less than about 6 inches (15 cm) of water column), to resist causing one or more suction-induced pressure ulcers in the bladder wall.

The instant urine drain system provides an important advance in the art of measuring urine output of a medical patient. The charge-volume of fluid in the conduit between the catheter 104 and end 118 (effectively the location of the siphon-break) can be determined. Such charge-volume can be supplied by the patient, or infused by medical personnel through an appropriate fluid coupling device (e.g., a 3-way valve, not illustrated, or a sample port associated with the catheter), disposed in association with the catheter 104. Assuming a small negative pressure (i.e., suction) is imposed by suitable location of the siphon-break elevation 132, and subsequent to filling such charge-volume, the patient's bladder will remain substantially empty. All urine produced by the patient will be removed substantially as it is produced, so the patient's fluid drain rate may be monitored with precision. The siphon conduit 102 remains filled with fluid, so no tiresome drain conduit manipulation is required to remove trapped pockets of fluid to ensure complete bladder draining prior to making a measurement. The change in volume contained in the drain container 138 may simply be determined at desired intervals of time to manually determine the patient's fluid discharge flow rate. Alternatively, an automated fluid flow rate determining device, such as the droplet counter disclosed in U.S. Pat. No. 6,640,649 issued Nov. 4, 2003 to Paz, et al., the contents of which are incorporated herein by this reference, may be used to determine the patient's discharge flow rate.

In any case, the instant drain assembly avoids need to manipulate the drain conduits to manually urge fluid motion in the drain conduit. The instant assembly can be configured and arranged to automatically ensure essentially complete evacuation of the bladder to reduce a nidus for infection. By avoiding fluid motion-inducing manipulations of the drain conduits, the instant assembly also avoids chance of inducing undesired retrograde urine flow into the bladder.

After being apprised of the instant disclosure, one of ordinary skill will be readily able to make the apparatus with materials and methods known in the art.

1. An apparatus, comprising:
a first conduit with at least a first lumen, the first lumen having a first cross-section area size and stretching between a first end and a second end of the first conduit, the first end being adapted to receive a fluid discharged from a urinary catheter, the second end being adapted to communicate the fluid toward a siphon-break device, the first lumen being configured to maintain plug-flow of a fluid flowing therethrough;
a siphon-break device, configured and arranged to resist plug-flow of the fluid flowing there-through, disposed in fluid communication with the second end of the first conduit; and
a second conduit arranged to communicate the fluid from downstream of the siphon-break device toward a container in which to receive the fluid; wherein the second conduit is configured and arranged to resist coupling the potential energy of fluid flowing downstream from the siphon-break device from fluid flowing in the first lumen.

2. The apparatus according to claim 1, wherein the second conduit comprises a second lumen having a second cross-section area that is sized larger than the first cross-section area size, and is configured and arranged in space effective to maintain particle-flow of the fluid downstream from the siphon-break device.

3. The apparatus according to claim 1, wherein an effective length of the second conduit adjusts to accommodate a plurality of elevations between a siphon-break location and the container to permit disposing the siphon-break device at approximately the same elevation as a subject's bladder while providing an uninterrupted downward-sloping drain path toward the container.

4. The apparatus according to claim 3, wherein the second conduit comprises a telescopic structure adapted for extension along an axis effective to lengthen a drain path between the siphon-break device and the container while providing an uninterrupted downward-sloping drain path toward the container.

5. The apparatus according to claim 3, wherein the second conduit comprises a stretch of conduit coiled about an axis and adapted for extension along the axis effective to lengthen an effective drain path between the siphon-break device and the container while providing an uninterrupted downward-sloping drain path toward the container.

6. The apparatus according to claim 3, wherein the second conduit comprises a serpentine labyrinth of substantially in-plane curved flow sections adapted for extension along an axis by changing a radius of curvature of at least one of the curved flow sections effective to lengthen an effective drain path between the siphon-break device and the container while providing an uninterrupted downward-sloping drain path toward the container.

7. The apparatus according to claim 1, wherein:
the siphon-break device is configured and arranged in harmony with the first conduit to visually indicate a corner approximately corresponding to the location of the siphon-break; and further comprising:
an anchor effective to maintain the corner in approximate registration with a top surface edge of a bed on which the apparatus may be installed.

8. The apparatus according to claim 1, wherein:
a discharge end of the second conduit is placed in fluid communication with the container; and
anchoring structure associated with the container is configured to permit making a vertical adjustment of the container with respect to suspension structure associated with a bed effective to facilitate disposing the siphon-break device at a desired elevation with respect to the bladder.

9. The apparatus according to claim 1, wherein the first conduit comprises a stretch coiled about an axis and adapted for extension along the axis effective to lengthen an effective drain path between the first end and the second end.

10. The apparatus according to claim 1, wherein the first conduit comprises a serpentine labyrinth of substantially in-plane curved flow sections adapted for extension by changing a length of lumen stretched between first and second of the curved flow sections effective to lengthen an effective drain path between the first end and the second end.

11. The apparatus according to claim 10, wherein the first conduit comprises a serpentine labyrinth of substantially in-plane curved flow sections adapted for extension by changing a radius of curvature of at least one of the curved flow sections effective to lengthen an effective drain path between the first end and the second end.

12. The apparatus according to claim 10, further comprising:
a hose management tool adapted to confine the serpentine labyrinth to a desired location in space, the tool being structured to permit transverse installation of a portion of the first conduit between gripping structure of the tool and configured for attachment to structure associated with a bed.

13. The apparatus according to claim 1, wherein a cross-section shape of the first lumen comprises at least one internally projecting arm configured to resist separation of a meniscus from the lumen wall.

14. The apparatus according to claim 1, further comprising:
a pressure indicator disposed in proximity to the siphon-break device and in fluid communication with the first conduit.

15. A method for using the apparatus according to claim 1 to measure the fluid output of a subject, the method comprising:
installing a urinary catheter in fluid communication with the bladder of the subject;
coupling the first end of the first conduit in fluid communication with a discharge end of the catheter;
disposing the siphon-break device at an elevation with respect to the bladder effective to induce a substantially constant pressure having a value selected from between about positive 1 inch (2.5 cm) water column and about negative 6 inches (15 cm) water column at a drain entrance of the catheter; and
placing a discharge from the siphon-break device into fluid communication with structure adapted to measure fluid flow rate.

16. The method according to claim 15, wherein the structure adapted to measure fluid flow rate comprises an automated fluid flow rate determining device.

17. The method according to claim 15, wherein:
the structure adapted to measure fluid flow rate comprises a container adapted to indicate a volume of fluid held therein; and wherein the method further comprises:
waiting for a first period of time during which a charge-volume at least fills a drain conduit disposed between the bladder and the siphon-break device, and determining a first volume contained in the container at the conclusion of the first period of time;
waiting for a second period of time, and determining a second volume contained in the container at the conclusion of the second period of time; and
calculating a fluid flow rate using the difference between the first and second volumes divided by the second period of time.

18. The method according to claim 15, wherein the structure adapted to measure fluid flow rate comprises a container adapted to indicate a volume of fluid held therein;
and wherein the method further comprises:
establishing a fully charged condition in the first conduit and ensuring that the container is empty;
waiting for a first period of time during which a first volume is discharged into the container;
determining the amount of first volume discharged;
calculating the volumetric flow rate by dividing the amount of first volume by the first period of time; and
emptying the first volume from the container into a downstream bulk container.

19. The method according to claim 15, further comprising:
measuring bladder pressure to infer abdominal pressure while permitting uninterrupted flow of fluid draining from the bladder.

20. A method for placing a urinary catheter in fluid communication with a drain container, the method comprising:
installing the urinary catheter in position to drain a patient's bladder;
placing a first end of a siphon conduit into fluid communication with a discharge end of the catheter, the siphon conduit being constructed to maintain plug-flow of a fluid flowing therethrough;
routing the siphon conduit along a path to dispose the siphon conduit discharge end elevated with respect to the drain container and at an elevation in general agreement with the bladder;
placing a first end of a siphon-break device in fluid communication with the discharge end, the siphon-break device being constructed to resist plug-flow of a fluid flowing therethrough; and
placing a second end of the siphon-break device in fluid communication with the drain container.

21. The method according to claim 20, further comprising:
anchoring structure associated with the siphon-break device effective to dispose a siphon-break location at an elevation between about 1 inch (2.5 cm) above, to about 4 inches (10 cm) below, the nominal elevation of the bladder.

22. The method according to claim 20, further comprising:
anchoring structure associated with the siphon-break device effective to maintain a consistent negative pressure through the catheter for operable effect of the negative pressure inside the bladder subsequent to complete charging of the siphon conduit.

23. The method according to claim 22, wherein the negative pressure has a value selected from between about ½ inch (1.27 cm) and about 6 inches (15 cm) of water column.

24. An apparatus for draining a urinary catheter installed in a medical patient's bladder effective to maintain the bladder in a substantially empty state, the apparatus comprising:
a siphon conduit adapted to maintain plug-flow of a fluid flowing therethrough, a first end of the siphon conduit being adapted to receive a fluid discharged from the urinary catheter, a second end of the siphon conduit being adapted for fluid communication toward a siphon-break device;
a siphon-break device disposed in fluid communication with the second end, and operable to define a drain path for fluid received from the siphon conduit, extending toward a container in which to receive the fluid, the siphon-break device further being effective to decouple the potential energy of fluid flowing along the drain path from fluid in the siphon conduit; and
anchor structure operable to dispose a siphon-break location, caused by operation of the siphon-break device on fluid discharged from the siphon conduit, at a desired and substantially fixed elevation with respect to a nominal position of the bladder.

25. The apparatus according to claim 24, wherein:
the anchor is configured for attachment to bedclothes effective to dispose the siphon-break location at an elevation effective to induce a substantially constant pressure, having a value selected from between about positive 1 inch (2.5 cm) water column and about negative 6 inches (15 cm) water column, at a drain entrance of the catheter.

26. The apparatus according to claim 24, wherein:
a portion of the apparatus, in proximity to the siphon-break device, is structured to define a shape that is complementary to the edge of a bed and effective to facilitate installation of the siphon-break device at a desired elevation.
